(19) United States
(12) Patent Application Publication
(10) Pub. No.: US 2021/0383925 A1
(43) Pub. Date: Dec. 9, 2021

(54) SYSTEMS FOR ADAPTIVE HEALTHCARE SUPPORT, BEHAVIORAL INTERVENTION, AND ASSOCIATED METHODS

(71) Applicant: INFORMED DATA SYSTEMS INC. d/b/a ONE DROP, New York, NY (US)

(72) Inventors: Ydo Wexler, Haifa (IL); Daniel R. Goldner, Minnetonka, MN (US); Jeffrey Dachis, Brooklyn, NY (US)

(21) Appl. No.: 17/338,586

(22) Filed: Jun. 3, 2021

Related U.S. Application Data
(60) Provisional application No. 63/034,331, filed on Jun. 3, 2020.

(51) Int. Cl. G16H 50/20; G16H 40/67; G16H 10/60; G16H 50/70; G06N 3/08; G09B 19/00

(52) U.S. Cl. CPC G16H 50/20; G16H 40/67; G16H 10/60; G16H 50/30; G06N 3/08; G09B 19/00; G16H 50/70

(57) ABSTRACT

Systems and methods for biomonitoring and personalized healthcare are disclosed herein. The method can include obtaining new data and accessing one or more user history items regarding a user; estimating a state of the user; identifying and executing an action for affecting a response of the user in assisting the user adjust a user behavior; and updating a model based on the response of the user.

### CROSS REFERENCE TO RELATED APPLICATION

This application claims priority to U.S. Provisional Patent Application No. 63/034,331, filed Jun. 3, 2020, the contents of which is incorporated herein by reference in its entirety.

### TECHNICAL FIELD

This disclosure relates generally to personalized healthcare and, in particular, to systems and methods for biomonitoring and healthcare guidance.

### BACKGROUND

Many individuals suffer from chronic health conditions, such as diabetes, pre-diabetes, hypertension, hyperlipidemia, and the like. For example, diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Diabetes affects a significant percentage of the world's population. Timely and proper diagnoses and treatment are essential to maintaining a relatively healthy lifestyle for individuals with diabetes. Application of treatment typically relies on accurate determination of glucose concentration in the blood of an individual at a present time and/or in the future. However, conventional health monitoring systems may be limited in availability or accessibility. Thus, there is a need for improved systems and methods for biomonitoring and/or providing personalized healthcare recommendations or information for the treatment of diabetes and other chronic conditions.

### BRIEF DESCRIPTION OF THE DRAWINGS

FIG. 1 is a schematic diagram illustrating an exemplary computing environment in which a healthcare guidance system operates, in accordance with embodiments of the present technology.

FIG. 2 is a diagram illustrating a representative deep learning neural network in accordance with embodiments of the present technology.

FIG. 3A—FIG. 3B are graphs illustrating example receptivity functions in accordance with some embodiments of the present technology.

FIG. 4 is a graph illustrating an example calculation of a mean and variance for a number of steps of users of a step-tracking software application in accordance with some embodiments of the present technology.

FIG. 5A-FIG. 5C are graphs illustrating comparisons between effects of using trained and random models in different simulations in accordance with some embodiments of the present technology.

FIG. 6 is an example flow diagram for determining a best action for a user in accordance with embodiments of the present technology.

FIG. 7A-7C illustrate example prompts and reinforcements output by the healthcare guidance system configured in accordance with embodiments of the present technology.

FIG. 8 is a schematic block diagram of a computing system or device configured in accordance with embodiments of the present technology.

FIGS. 9-10 are schematic diagrams illustrating exemplary computing environments in which a healthcare guidance system operates, in accordance with embodiments of the present technology.

### DETAILED DESCRIPTION

The present technology generally relates to systems and methods for biomonitoring and providing personalized healthcare. In some embodiments, a healthcare guidance system is configured to obtain and analyze real-time biomonitoring data and provide adaptive healthcare support to guide a patient user in completing health-related tasks to manage and/or improve a condition (e.g., diabetes, pre-diabetes, hypertension, hyperlipidemia, etc.). The healthcare guidance system can guide the patient using goals, prompts, alerts, reminders, reinforcements, feedback, etc. The healthcare guidance system can continuously or periodically update and/or adapt the guidance based on data from the patient user as well as data from a plurality of other patients (via, e.g., crowdsourcing mechanisms). The system can use the support to guide individuals toward behavioral interventions that are likely to improve their health outcomes.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

### Systems for Biomonitoring and Healthcare Guidance

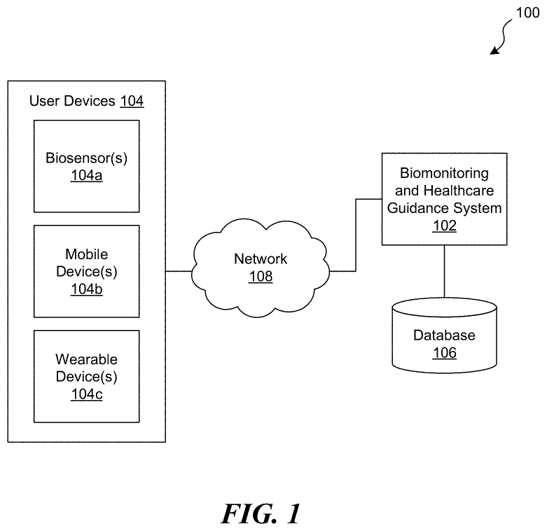

FIG. 1 is a schematic diagram of an exemplary computing environment in which a biomonitoring and healthcare guidance system 100 (“system 100”) operates, in accordance with embodiments of the present technology. As shown in FIG. 1, the system 100 can include one or more user devices 104 operably coupled to a biomonitoring and healthcare guidance system in the form of analyzing devices 102. The system 100 can further include to at least one database or storage component 106 (“database 106”) coupled to the analyzing devices 102 and/or the user devices 104. The various devices may be coupled to each other via a network 108. The system 100 can include processors, memory, and/or other software and/or hardware components configured to implement the various methods described herein. For example, the system 100 can be configured to monitor a patient/user health state and provide adaptive healthcare support, as described in greater detail below.

For example, the user devices 104 can obtain biometric data, such as temperature, heartrate, blood pressure, blood glucose level or the like, and/or spatial data, such as location, acceleration, velocity, orientation, a change thereof over time, or the like. The user devices 104 can also obtain contextual data, such as user calendar (including, e.g., event name or category, location, date, time, participant, etc.), that may be used to categorize other obtained data. For example, the contextual data may be used to categorize the data into user activities or user health states.

The health state can be any status, condition, parameter, etc. that is associated with or otherwise related to the patient's health. In some embodiments, the system 100 can be used to identify, manage, monitor, and/or provide recommendations relating to diabetes, hypoglycemia, hyperglycemia, pre-diabetes, hypertension, hyperlipidemia, ketoacidosis, liver failure, congestive heart failure, hypoxia, kidney function, intoxication, dehydration, hyponatremia, shock, sepsis, trauma, water retention, bleeding, endocrine disorders, asthma, lung conditions, muscle breakdown, malnutrition, body function (e.g., lung functions, heart functions, etc.), physical performance (e.g., athletic performance), anaerobic activity, weight loss/gain, nutrition, wellness, mental health, focus, effects of medication, medication levels, health indicators, and/or user compliance. In some embodiments, the system 100 receives input data and performs monitoring, processing, analysis, forecasting, interpretation, etc. of the input data in order to generate user reports, behavior goals, instructions, notifications, recommendations, support, and/or other information to the patient that may be useful for self-care of diseases or conditions, such as chronic conditions (e.g., diabetes (type 1 and type 2), pre-diabetes, hypertension, hyperlipidemia, etc.).

The input data for the system 100 can include health-related information, contextual information, and/or any other information relevant to the patient's health state. For example, health-related information can include levels or concentrations of a biomarker, such as glucose, electrolytes, neurotransmitters, amino acids, hormones, alcohols, gases (e.g. oxygen, carbon dioxide, etc.), creatinine, blood urea nitrogen (BUN), lactic acid, drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.), stress level data (e.g., cortisol and/or other chemical indicators of stress levels, perspiration), a1c data, etc. Health-related information can also include medical history data (e.g., weight, age, sleeping patterns, medical conditions, cholesterol levels, disease type, family history, patient health history, diagnoses, tobacco usage, alcohol usage, etc.), diagnostic data (e.g., molecular diagnostics, imaging), medication data (e.g., timing and/or dosages of medications such as insulin), personal data (e.g., name, gender, demographics, social network information, etc.), and/or any other data, and/or any combination thereof. Contextual information can include user location (e.g., GPS coordinates, elevation data), environmental conditions (e.g., air pressure, humidity, temperature, air quality, etc.), and/or combinations thereof.

In some embodiments, the analyzing devices 102 receives the input data from the user devices 104. The user devices 104 can be any device associated with a patient or other user, and can be used to obtain healthcare information, contextual information, and/or any other relevant information relating to the patient and/or any other users or patients (e.g., appropriately anonymized patient data). In the illustrated embodiment, for example, the user devices 104 can include at least one biosensor 104a (e.g., blood glucose sensors, pressure sensors, heart rate sensors, sleep trackers, temperature sensors, motion sensors, or other biomonitoring devices), at least one mobile device 104b (e.g., a smartphone or tablet computer), and, optionally, at least one wearable device 104c (e.g., a smartwatch, fitness tracker). In other embodiments, however, one or more of the devices 104a-c can be omitted and/or other types of user devices can be included, such as computing devices (e.g., personal computers, laptop computers, etc.). Additionally, although FIG. 1 illustrates the biosensor(s) 104a as being separate from the other user devices 104, in other embodiments the biosensor(s) 104a can be incorporated into another user device 104.

The biosensor 104a can include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. In some embodiments, the biosensor 104a is or includes a blood glucose sensor. The blood glucose sensor can be any device capable of obtaining blood glucose data from the patient, such as implanted sensors, non-implanted sensors, invasive sensors, minimally invasive sensors, non-invasive sensors, wearable sensors, etc. The blood glucose sensor can be configured to obtain samples from the patient (e.g., blood samples) and determine glucose levels in the sample. Any suitable technique for obtaining patient samples and/or determining glucose levels in the samples can be used. In some embodiments, for example, the blood glucose sensor can be configured to detect substances (e.g., a substance indicative of glucose levels), measure a concentration of glucose, and/or measure another substance indicative of the concentration of glucose. The blood glucose sensor can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor can include other capabilities, such as processing, transmitting, receiving, and/or other computing capabilities. In some embodiments, the blood glucose sensor can include at least one continuous glucose monitoring (CGM) device or sensor that measures the patient's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

In some embodiments, some or all of the user devices 104 may be configured to continuously obtain any of the above data (e.g., health-related information and/or contextual information) from the patient over a particular time period (e.g., hours, days, weeks, months, years). For example, data can be obtained at a predetermined time interval (e.g., in the order of seconds, minutes, or hours), at random time intervals, or combinations thereof. The time interval for data collection can be set by the patient, by another user (e.g., a physician), by the analyzing devices 102, or by the user device 104 itself (e.g., as part of an automated data collection program). The user device 104 can obtain the data automatically or semi-automatically (e.g., by automatically prompting the patient to provide such data at a particular time), or from manual input by the patient (e.g., without prompts from the user device 104). The continuous data may be obtained by the system 100 (e.g., collected at the analyzing devices 102) at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmit the data to the analyzing devices 102.

The user devices 104 can obtain any of the above data and can provide output in various ways, such as using one or more of the following components: a microphone (either a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the analyzing devices 102) before being used.

In some embodiments, any of the above data can be queried by one or more of the user devices 104 from one or more databases (e.g., the database 106, a third-party database, etc.). The user device 104 can generate a query and transmit the query to the analyzing devices 102, which can determine which database may contain requisite information and then connect with that database to execute a query and retrieve appropriate information. In other embodiments, the user device 104 can receive the data directly from the third-party database and transmit the received data to the analyzing devices 102, or can instruct the third-party database to transmit the data to the analyzing devices 102. In some embodiments, the analyzing devices 102 can include various application programming interfaces (APIs) and/or communication interfaces that can allow interfacing between user devices 104, databases, and/or any other components.

Optionally, the analyzing devices 102 can also obtain any of the above data from various third party sources, e.g., with or without a query initiated by a user device 104. In some embodiments, the analyzing devices 102 can be communicatively coupled to various public and/or private databases that can store various information, such as census information, health statistics (e.g., appropriately anonymized), demographic information, population information, and/or any other information. Additionally, the analyzing devices 102 can also execute a query or other command to obtain data from the user devices 104 and/or access data stored in the database 106. The data can include data related to the particular patient and/or a plurality of patients or other users (e.g., health-related information, contextual information, etc.) as described herein.

The database 106 can be used to store various types of data obtained and/or used by the system 100. For example, any of the above data can be stored as user history 124 in the database 106. The database 106 can also be used to store data generated by the system 100, such as previous predictions or forecasts produced by the system 100. In some embodiments, the database 106 includes data for multiple users, such as a plurality of patients (e.g., at least 50, 100, 200, 500, 1000, 2000, 3000, 4000, 5000, or 10,000 different patients). The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the analyzing devices 102 and/or the user devices 104. The tables can store different types of information (e.g., one table can store blood glucose measurement data, another table can store user health data, etc.), where one table can be updated as a result of an update to another table.

In some embodiments, one or more users can access the system 100 via the user devices 104, e.g., to send data to the analyzing devices 102 (e.g., health-related information, contextual information) and/or receive data from the system 100 (e.g., predictions, notifications, recommendations, instructions, support, etc.). The users can be individual users (e.g., patients, healthcare professionals, etc.), computing devices, software applications, objects, functions, and/or any other types of users and/or any combination thereof. For example, upon obtaining any of the input data discussed above, the user device 104 can generate an instruction and/or command to the analyzing devices 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the system 100 to perform an analysis of data stored in the database 106 and/or inputted via the user device 104.

As discussed further below, the analyzing devices 102 can analyze the obtained input data, including historical data, current real-time data, continuously supplied data, and/or any other data (e.g., using a statistical analysis, machine learning analysis, etc.), and generate output data. The output data can include predictions of a patient's health state, interpretations, recommendations, notifications, instructions, support, and/or other information related to the obtained input data. The analyzing devices 102 can perform such analyses at any suitable frequency and/or any suitable number of times (e.g., once, multiple times, on a continuous basis, etc.). For example, when updated input data is supplied to the analyzing devices 102 (e.g., from the user devices 104), the analyzing devices 102 can reassess and update its previous output data, if appropriate. In performing its analysis, the analyzing devices 102 can also generate additional queries to obtain further information (e.g., from the user devices 104, the database 106, or third party sources). In some embodiments, the user device 104 can automatically supply the analyzing devices 102 with such information. Receipt of updated/additional information can automatically trigger the analyzing devices 102 to execute a process for reanalyzing, reassessing, or otherwise updating previous output data.

In some embodiments, the analyzing device 102 is configured to analyze the input data and generate the output data using one or more machine learning models 122. The machine learning models 122 can include supervised learning models, unsupervised learning models, semi-supervised learning models, and/or reinforcement learning models generated by one or more modeling engines 112. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest).

Although FIG. 1 illustrates a single set of user devices 104, it will be appreciated that the analyzing devices 102 can be operably and communicably coupled to multiple sets of user devices, each set being associated with a particular patient or user. Accordingly, the system 100 can be configured to receive and analyze data from a large number of patients (e.g., at least 50, 100, 200, 500, 1000, 2000, 3000, 4000, 5000, or 10,000 different patients) over an extended time period (e.g., weeks, months, years). The data from these patients can be used to train and/or refine one or more machine learning models implemented by the analyzing devices 102, as described below.

The analyzing devices 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the analyzing devices 102 as being directly connected to the database 106 without the network 108, in other embodiments the analyzing devices 102 can be indirectly connected to the database 106 via the network 108. Moreover, in other embodiments one or more of the user devices 104 can be configured to communicate directly with the system 100 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices. For example, although FIG. 1 illustrates the analyzing devices 102 as being a single component, in other embodiments the analyzing devices 102 can be implemented across a plurality of different hardware components at different locations.

In some embodiments, the analyzing devices 102 may include a state estimator 114 configured to estimate a user context or a user health state. The state estimator 114 can use the above-described input data to determine a current or ongoing activity or state of the user. Similarly, the state estimator 114 can analyze the above-described input data to predict a future activity or health state of the user. The state estimator 114 can use corresponding machine learning models 122 to generate the estimated user states. For example, the state estimator 114 can use the models 122 to predict based on the current state and the user history 124 that blood glucose levels will reach a threshold level at a time when the user will likely be incapacitated (e.g., sleeping or intoxicated). In some embodiments, the state estimator 114 can generate activity predictions for a predetermined future duration based on the obtained data. The state estimator 114 can start from a current health state (e.g., blood glucose level) and extrapolate or derive future health states according to the activity predictions. The system 100 can use the future health states to determine and recommend one or more user actions that may be implemented between now and a future time to avoid the thresholding health states.

In some embodiments, the system 100 can evaluate an effectiveness in varying a timing and/or a magnitude (e.g., language selection, award amount, etc.). For example, the analyzing devices 102 can use one or more of the models 122 configured to represent user preferences or motivations that contribute to complying with or implementing the recommended action. As an illustrative example, some users may be more motivated by urgent language and/or immediate consequences as indicated by the user history 124 and/or behaviors of other users having a shared trait. The response models for such patient user may be configured to award higher scores to more dramatic or urgent wording/messages, recommended actions having higher physical demands, recommended actions requiring shorter durations, and/or recommendation timings closer to the thresholding event. Other users may be more motivated by reducing pain or physical exertion. The response models for such users may be configured to award higher scores to wording/messages that emphasize reduction of negative consequences, recommended actions having lower physical demands, recommended actions requiring longer durations, and/or recommendation timings further away from the thresholding event. Details regarding the state estimator 114 and the corresponding recommendations are described below.

### Methods for Biomonitoring, Healthcare Guidance, and Adaptive Healthcare Support

In some embodiments, the healthcare guidance systems described herein (e.g., the system 100 of FIG. 1) are configured to provide adaptive healthcare support for an individual (e.g., a patient having a disease or condition and a user of the system 100). The adaptive health care support can include, for example, one or more adaptive behavioral interventions, e.g., individualized interventions that vary support based on a person's evolving needs. Digital technologies can be used to allow these adaptive interventions to function at scale. For example, a user device (e.g., user devices 104 of FIG. 1) can display output (e.g., messages, prompts, reinforcements etc.), output audible alerts, or other output. Adaptive interventions may produce better results compared with static interventions related to health outcomes. In some embodiments, one or more elements of an adaptive intervention are iteratively improved via data-driven testing (e.g., optimization), which may improve the likelihood of successfully helping individuals to meet and maintain behavioral targets.

Various methods can be used to calculate adjustments to an intervention. For example, in some embodiments, the system uses an adaptive support model to determine how to adjust the individual's intervention from day to day. The model can be trained on pooled data from a large number of individuals. The system can use a reinforcement learning framework to perform the prediction-and-optimization (“control systems engineering”) task.

In some embodiments, the system optimizes two forms of support for a single target behavior: prompt messages and reinforcement messages. Alternatively or additionally, the system may further utilize warnings or messages having varying degrees of urgency or impact. The target behavior can be, for example, a single action (a task) which may be performed regularly, such as taking medication, checking blood sugar or blood pressure, or similar discrete, repeated actions. The system can receive data on an individual's past performance of the task, past prompt and reinforcing messages sent to the individual, and/or related information about the individual. The system can input that information to a trained model, which determines when to send prompt or reinforcement messages so as, over time, to bring the individual's rate of task completion toward a target rate.

In embodiments where the task is a discrete action, each day can be divided into a number of periods. At the start of each period, the model can calculate whether or not to send a prompt message. During the period, the individual either performs the task, or does not. The model can calculate whether or not to send a reinforcement message if the individual performs the task during the period.

From day to day and over time, a single individual may change in their responsiveness to prompts and reinforcements. A prompt rate that was helpful at one point can become easy to ignore later on, or may become annoying. Different individuals may also react differently to prompts and reinforcements. Moreover, different individuals may react differently according to the urgency of the content, the type of communication (e.g., warnings or prompts), output format (e.g., audible or visual, emphasizing numbers or graphics, etc.), the timing of the message relative to a desired timing of the response, or the like. Accordingly, one goal of the optimization at each period can be to determine whether, for that individual at that time, a prompt, reinforcement, or both would be effective or not at getting the individual to do the task at the desired rate. Also, the optimization can determine the urgency, the type, the timing, etc. associated with delivering the message or recommendation.

In some embodiments, at each period, the goal of the calculations may not simply be to maximize the probability of task completion in the following period, but rather, to increase or maximize the expected discounted rate of task completion into the future. In such embodiments, messaging that results in high rates of task completion for a short time but then no further task completion may be considered less successful than messaging that results in building the individual's task completion rate up to the desired level and maintaining it there for a long period of time. In short, the model's decisions can be aimed at optimizing the overall task completion by the individual over time, with more emphasis on task adherence in the near future.

In some embodiments, an adaptive support model can be configured to learn from a multitude of users while keeping track of the messages and behavior of each user over time, in order to serve them with the optimal messages at every time slot. The model may use a definition of (a) a vector describing the state (e.g., the health state or the current activity) of an individual at a given time, (b) a list of possible actions the system can take, and/or (c) a reward function. Each of these elements is described in detail below.

For a particular individual, inputs can include the individual's history of task completions, and the history of supports (e.g., prompts and reinforcements) delivered to the individual. For example, this information can be recorded as follows: for each period of each past day, record a prompt as 1, no prompt as 0; a task completion as 1, no task completion as 0; a reinforcement as 1 and no reinforcement as 0.

Inputs can be summarized in various ways to allow for comparison between histories of one individual at one time versus the same individual at a different time, or versus a different individual's history. For example, the system can calculate three exponentially weighted moving averages, one for each sequence of 1's and 0's (e.g., prompts, completions, and reinforcements), representing recent average prompt frequency, recent average task completion frequency, and recent average reinforcement frequency.

Additional further values can be generated from the history of task completions, prompts and reinforcements. For example, propensity and/or sensitivity can be generated. Propensity can be the base probability of completing a task, effectively representing the effects of all factors not explicitly represented in the model. Sensitivity can be the strength and direction of the effects of reinforcements on an individual. Sensitivities cam be ranged from “receptive” (e.g., reinforcing strongly increased the probability of task completion) to “habituated” (e.g., reinforcing had little effect on task completion) to “sensitized” (e.g., reinforcing strongly decreased task completion.) Both propensity and sensitivity can be assumed to change over time. For example, a user may be receptive to reinforcements for upcoming activities to be performed and sensitized to reinforcements associated with a recently completed actions. User states, events, and activities can be correlated to determine relationships between the data, thereby enabling the system to forecast propensity and/or sensitivity.

One or more probability functions can be used to express the current probability of the individual completing a task according to a function of the recent average prompt frequency, recent average task completion frequency, recent average reinforcement frequency, current propensity, and/or current sensitivity. The system can then generate values of propensity and sensitivity as the maximum likelihood estimators given the data and a probability function. A set of probability functions can be associated with the user to generate values of propensity and sensitivity for the user state, predicted state, etc. For example, a set of probability functions can be used when a user is receptive to receiving prompts and another set of a set of probability functions can be used at night when the user is typically not receptive to receiving prompts.

At any point in time (any period of any day), various parameters, such as recent average prompt frequency, recent average task completion frequency, recent average reinforcement frequency, estimated propensity, and/or estimated sensitivity, can provide the state vector for the person. Optionally, the state vector can be expanded to include other types of input information, as discussed further below.

In some embodiments, the system determines, for each period, which of the following actions to take: The actions can include not sending a message, sending a prompt message, sending a reinforcement message if the individual performs a task, and sending both a prompt message and a reinforcement message in response to the individual performing the task associated with the prompt message. The system may further determine content type, presentation type, and/or delivery timing for the prompt and/or reinforcement messages.

The system can choose actions to maximize a reward, e.g., based on the individual's predicted future task completions. A task completion can be awarded a positive reward, and a period in which the task is not completed can receive a negative reward. The reward can be discounted (or decaying), meaning that task completions predicted in the near future can be valued more highly than later task completions.

Accordingly, the model can be a mapping M(s, a)→R where s is a state vector as described before, and a is an action out of the possible actions. R can be a real number that represents the expected sum of decaying reward as estimated by the model. Given a state s for the user, the model can estimate the reward for each of the possible actions. In some embodiments, the system typically chooses and enacts the action that maximizes this reward. However, a very small percentage of the time, the system may instead choose a random action. This can allow the system to continue to learn how the individual is responding to different actions at different times.

The model can be trained via the reinforcement-learning paradigm, e.g., where a procedure adjusts the mapping from states to actions to arrive at an optimal policy, which maximizes the expected sum of decaying reward. In some embodiments, deep reinforcement learning can be used. Deep reinforcement learning can use a deep neural network model to learn a mapping from states to the expected rewards. At every iteration, when given a state s, the model can predict the expected sum of decaying rewards, using the predetermined decay rate γ for every action. After choosing an action a, the model can see a reward r for that action. Given that the predicted vector of rewards was \hat{R}, with \hat{r}ₗ as the reward for action i, the model can feed the deep neural network model with an example whose input is s and output is a modified reward vector R that equals \hat{R} everywhere but in the entry j of the action taken. The new value for this entry can be set to be r+γ^{\hat{r}}_{J}. For example, the supervised model can be a deep neural network with 6 layers of varying size, with softmax activation for the last layer and ADAM optimizer.

In other embodiments, however, other deep-learning models can be used. For example, Deep-Q learning (“DQN” or “a Deep-Q network”) can be used. In a DQN, an optimal policy can be learned by a deep neural network model. Q learning utilizes an approach where the expected sum of decaying rewards is maximized using the following formula: Qs,a=r(s,a)+maxa′ Qs′,a′ for a state s and action a, where is a decay factor close to one, r(s,a) is the immediate reward that may be collected from performing action a when at state s, and s′ is the new state to which the environment transitions to. This formulation is most suitable for deterministic environments and a similar formulation is available for stochastic ones.

In a DQN, a deep neural network model acts at the optimizer of the Q function. Over time, the deep neural network model learns to approximate the Q function by training using more or more training examples, where the state s is given as an input and value of the Q function for all possible actions is given as a known output (e.g., supervised training).

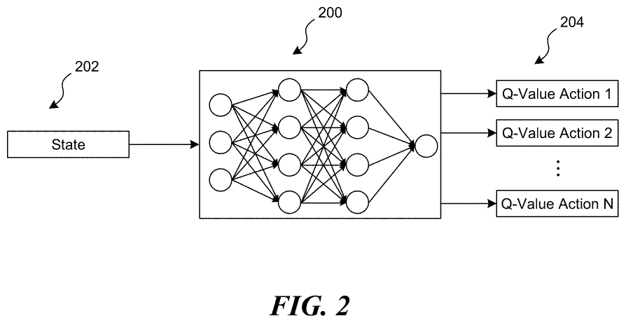

An example of a deep neural network model 200 is illustrated in FIG. 2. The example deep neural network model 200 can receive a current state 202 of the patient. The deep neural network model 200 can then train a reinforcement learning (“RL”) model by observing a current state and receiving, from a deep network, an assessment of Q values (e.g., scores) for different actions 204 associated with the state. From the different actions, an action can be selected based on the best possible Q value for each action. This selection can be performed stochastically based on the estimated Q values for each action. A possible reward for transitioning to a new state can also be identified. The deep neural network model 200 then uses this data as an example to train the model, with the state as the input, the estimated Q values as the output for all actions that were not selected, and Q=r+Qes,a where r is the observed reward, and Qes,a is the estimated Q-value for the selected action. In the context of ASM, a DQN-RL model learns a policy that maps from a state, which summarizes what we know about the user, into estimated Q-values, one for each possible action, where the rewards are behavior-related, and represent the level to which a user succeeds in the task.

For example, to help one person develop the habit of checking the blood glucose level regularly, the actions available to the application at each iteration is to prompt the person or not to check the blood glucose level. For every iteration, the RL model observes a summarized history of the person's behavior and prompts, selects an action, and gets a reward of 1 if the person logs her the blood glucose level the next iteration and −1 if she doesn't. To help another person increase his daily step count over time, the actions available to the application at each iteration are to select 1000, 3000, or 7000 steps. Every iteration the RL model observes a distribution over the person's daily steps and the number of steps relative to the set goal, selects a number of steps as his daily goal for that iteration, and gets a reward that equals the number of steps he's taken.

Optionally, a replay memory can be used, which allows training of the deep neural network only every set amount of time slots, and repeats previous examples by randomly selecting from a memory that holds a set number of recently experienced previous periods.

In some embodiments, the model can prioritize increasing the task completion rate of individuals whose current rate (e.g., average task completion rate over a long time) is very low, over increasing the task completion rate for individuals whose rate is already higher. In such embodiments, the rewards can be set to be asymmetrical, where a negative reward (for not adhering to the task) is proportional to the prior probability with a fixed minimum penalty, while positive rewards are linearly inversely proportional to the prior probability with a fixed minimum. Accordingly, users with lower prior probability can get higher positive rewards, and users with a higher probability can get a reward that is not less than a fixed ratio from the maximal one.

In some embodiments, the model may be trained and used on data from a single individual, e.g., using many periods of observed experience. In other embodiments, however, the model can be trained on observed periods from multiple individuals (e.g., thousands of patients). In such embodiments, an individual who is new to the system can benefit, because the model will already have experience with people who have been in a similar state (e.g., a health state, an activity, a context, etc.). Likewise, someone who is in a state they have not been in before can still get the benefit of prior experience of others having similar states. Pooling data can help to generalize the model across individuals and/or across time.

The approaches described herein can also be configured to address the problem that, before any prompts or reinforcement messages are delivered, there is no historical record of states and task completions to use for training. This problem can be addressed by creating simulations of individuals. In some embodiments, each simulation can represent an individual (or group of similar individuals) with an individual propensity and sensitivity, and logic that causes those values to change over time. In a given time period, the simulated current probability of completing the task can be a function of the simulated individual's state (as defined above) and of the simulated individual's current values of propensity and sensitivities. A random draw with the current probability can be performed to determine whether the simulated individual then completed the task during that period.

The model can be trained by connecting the reinforcement learning system to the simulated individuals. The system can prompt and reinforce the simulated individuals as described above. In one non-limiting example, thousands of simulations were created, each iterating over 3000 periods, and used to train an adaptive support model with each iteration until convergence. After 1500 simulations, the model converged to an optimum from which there was little further change, even after 5000 more simulations. Across the population of simulated individuals, this optimum produced task completion rates an average of 3.5 times higher than those produced by randomly selecting actions, and 8 times higher for those simulated individuals whose initial frequency of task completion was lower than 2%. In some embodiments, the support, intervention, and/or assistance provided is not restricted to prompts and reinforcements. Supports can be or include any communication or activity through a user device, intended to improve or increase performance of the target behavior, including but not limited to a prompt or reminder to do the target behavior; a reinforcement (e.g., a message reacting to an individual's single performance of the target behavior); feedback (e.g., a message summarizing or otherwise reacting to the individual's observed record of performance of the behavior); and/or a short-term goal or challenge (e.g., a particular manifestation of the behavior to practice on a particular day).

Additionally, behaviors that can be supported with such a system are not restricted to discrete, periodic tasks. A behavior can be any habit, change of habit, or routine choice, including increasing (or decreasing) consumption of a particular kind of food, changing levels of daily physical activity, etc.

In addition, multiple target behaviors can be supported simultaneously, by defining the action space to include all actions supporting the different targets, and the reward function to be the sum (or weighted sum or other combination) of the reward functions for the individual behaviors. To allow for this general framework to support a multitude of behaviors, tasks and actions, a virtual user can be implemented. Each instantiation of a virtual user represents the estimated relevant summary and mechanism of a real user for a given task. A virtual user may correspond to a set of components: parameters, state, update functions, reward functions, and simulated behavior functions. Parameters are invariant values the moderate the dynamics of behavior for a user in a predetermined way. State includes an array of user values that are observable or estimable, and can change every step based on the actions from the software application and the behavior of the user. Update functions are used to update the state of the user and associated probability distributions for particular behaviors that are determined based on the state of the user, the parameters of the user, recent behaviors of the user, and the like. Reward functions are functions that determine if a reward should be given based on user behavior(s) and optionally the user's state. Simulated behavior functions return a probability distribution for behaviors based on the state of the user and a selected action. Additionally, in this framework, any external information that can be considered relevant to user behavior can be added to the state.

Different tasks are defined by different types of behaviors and different sets of actions. Therefore, different tasks require different instantiations of the virtual user. For example, multiple types of task-specific user classes can be implemented, each serving to promote a unique behavior.

A first example type of task specific user class can be a reinforced user class. Reinforced user classes are associated with a user that has a binary behavior (behaves or doesn't behave at every step) and responds to a binary action of reinforcement, where she can be reinforced only after behaving. The goals of utilizing the reinforced user class is to maximize user behavior over time. The reinforced user class is controlled by several forces, such as a probability of behaving decreasing by a fixed factor at each step, the probability of behaving converging at each step to a moving average of recent behaviors by an unknown factor of a gap between the two values, and/or a receptivity function defined by Equation 1.

$$- \frac{c}{( 1 + r )} ( \frac{2}{1 + exp ( - \frac{( x - t )}{K} )} - ( 1 + r ) ) . \qquad Equation 1$$

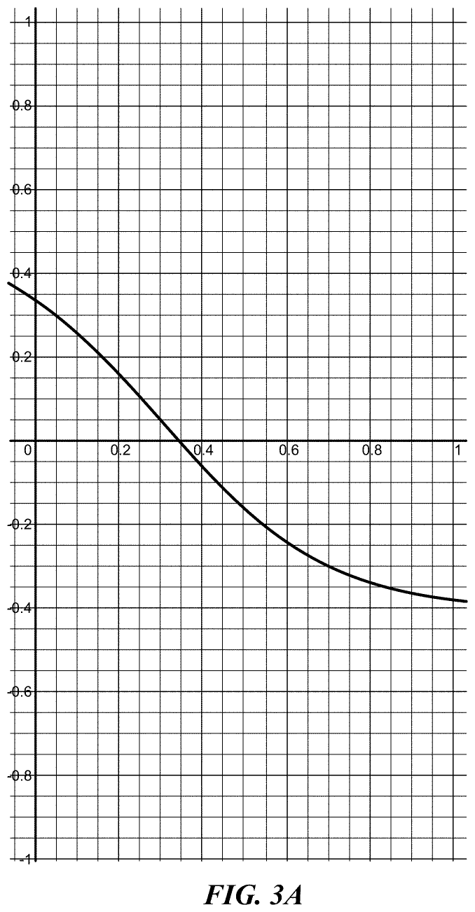

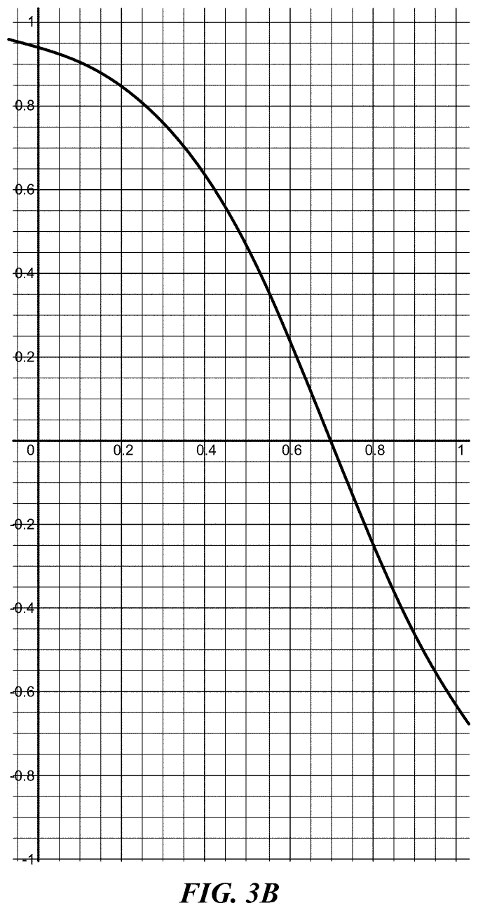

This function can be a function with an inverted sigmoid shape and source input values in the range (0, 1). K is a constant that determines the slope of the function, while the other variables are user parameters and hidden from the model. C can be a magnitude of a reinforcement effect, t determines an inflection for the function where the function turns from positive to negative, and r is a bias or shift towards positive output values. These values determine the shape of the receptivity function and are received from one or more user parameters. X is a fraction of times the user was reinforced recently, computed by dividing a moving average of the user being reinforced by a moving average of the user behaving. Under the assumption that, at first, users are less susceptible to frequent reinforcement, the threshold parameter t is initially set equal to 1. Every time a step is performed, the value for t is decreased by a predetermined value until a threshold value is reached. In some implementations, both the slope and threshold value can be controlled by additional parameters. Two examples of the function are shown in FIG. 3A and FIG. 3B. In FIG. 3A, the variables have values of c=0.5, r=0.1, and t=0.3. In FIG. 3B, the variables have values of c=1.0, r=0.0, and t=0.7.

A second example type of task specific user class can be a prompted user class. Prompted user classes are associated with a user that has a binary behavior (behaves or doesn't behave at every step) and responds to action with two binary values. The binary values can indicate if the user is prompted or reminded to perform the task in that step and/or indicate that the user should be reinforced after completing the task. Like the reinforced user class, the goal is to maximize the user's behavior over time. The forces that affect the prompted user class are similar to those of the reinforced user class. However, the prompted user class an additional effect from prompts can be computed. For prompted user classes, a receptivity function can be application to a fraction of recent prompts of the user, which can then be multiplied by a probability of a user performing a behavior if the behavior is negative and/or be multiplied by 1 minus the probability of the user performing the behavior if the behavior is positive. The resulting value of this multiplication then added to the probability of the user behaving at a given step. In some implementations, there is a threshold parameter for prompts that is different than the threshold value for governing reactions to reinforcement.

A third example type of task specific user class can be a steps user class. In a steps user class, a user can have behavior defined as a non-negative integer number selected from a set range of values, which represents a steps goal set by a software application. Examples of what the non-negative integer can represent include consumption of a particular food or nutrient, minutes of daily physical activity, number of “standing breaks” performed by the user (e.g., interruptions to sedentary behavior), number of mindfulness sessions performed by the user (e.g., breaks to stop and take several deep breaths), number of hours of sleep, or number of daily steps. The user can also have behavior defined by a binary value that represents whether the user should be reinforced if the steps goal is matched or exceeded. In a non-limiting example, a use case of the steps user class can include tracking a number of daily steps for a user. A prior probability of the number of steps a steps user takes is assumed to follow a normal distribution. The mean of the normal distribution is set to an unbiased empirical estimation of a recent number of steps as computed by a moving average of the number of steps. To assess a conditional probability distribution of steps the user is most likely to walk given the goal set for the user and any reinforcements the user has been given. A standard deviation that converges over time to an empirical standard deviation can also be set, and a mean of the distribution can be determined based on a set dynamic.

For ease of computation and speed, a clipped normal distribution is used, which is projected on a range within −1 and 1. This value describes the user's motivation and is used as a user parameter. In some implementations, the mean of this distribution can be a positive value, and at least one of the edges can be a negative value.

To compute a change in the distribution between steps, the probability of the user achieving the set goal (or the “feasibility” or “f”) is first determined, as the probability of achieving the goal given the distribution of the user's recent step counts. Based on the motivation distribution of the user, a user motivation (“m”) can also be calculated based on the feasibility, as follows: The motivation distribution can describe how success probability changes depending on the difference between the goal and the peak of the motivation distribution, i.e., goals which are much higher or much lower than the peak motivation goal can receive a negative motivation, where goals close to the peak motivation goal can result in positive motivation. The probability of the user meeting the set goal is then calculated as probability=feasibility plus motivation plus reinforcement receptivity. A new mean for the distribution can then be set as the current meant plus a fixed fraction of the z score of the calculated probability multiplied by the standard deviation of the step's distribution.

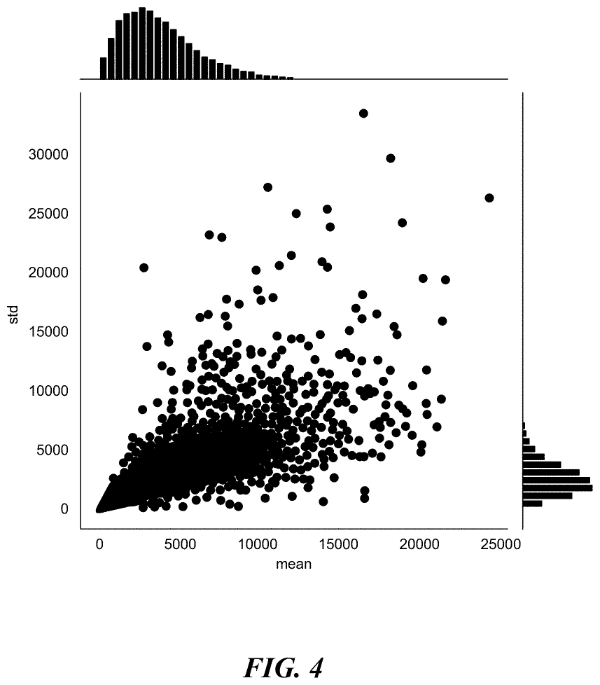

To train the model on users with realistic parameters, one or more databases can be accessed to obtain numbers of steps for users of a step-tracking software application. The mean and variance of the number of steps per user can be calculated. A graph showing this distribution calculation is shown in FIG. 4. Once the mean and variance of the distribution are calculated, a model can be fitted over the two variables. The model can then be used to determine the mean and standard deviation of random users. In some implementations, the model can be a Mixed Vine copula model.

To test the validity of the deep neural network 200 and to evaluate the effectiveness of the deep neural network 200 in supporting users towards beneficial behaviors, a model can be trained for each of the implemented user types. A simulation includes generating users with random parameters, and iterating for a set number of steps. In every step an action is chosen, the simulation samples a behavior from the distribution over behaviors as given by a simulated-behavior function of the user, and the state of the user is updated based on an update function.

The trained deep neural-network 200 can include multiple (e.g., five, six, seven) full layers of varying number of neurons, e.g., up to 256 neurons in a single layer. After each layer batch normalization may be performed, followed by a drop-out of a set (e.g., half) of the neurons. The networks for the reinforced user class and prompted user class can use softmax as the activation function, and the one for steps user class can use a simple linear output. For each of the user types the average behavior and the mean behavior probability of the user at the end of every episode can be compared with that of a model that draws actions at random from the actions space.

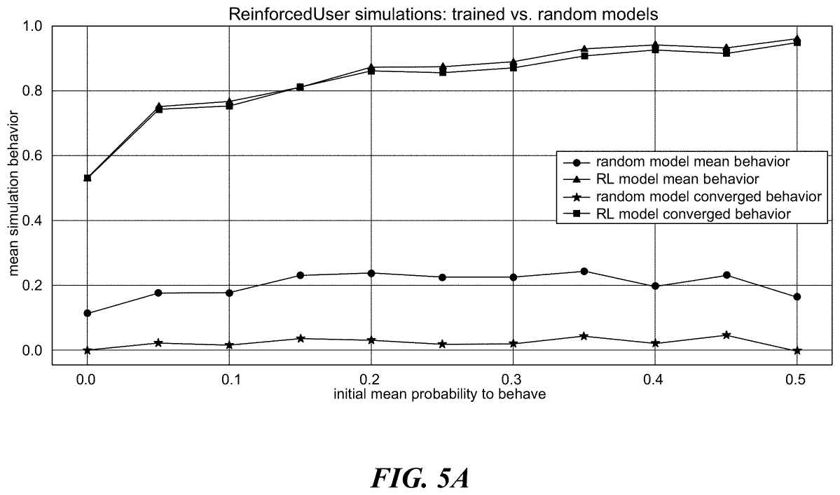

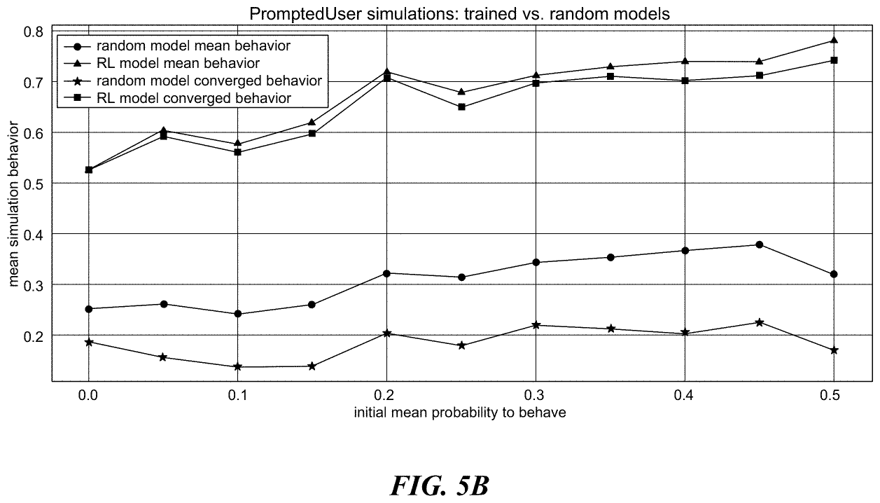

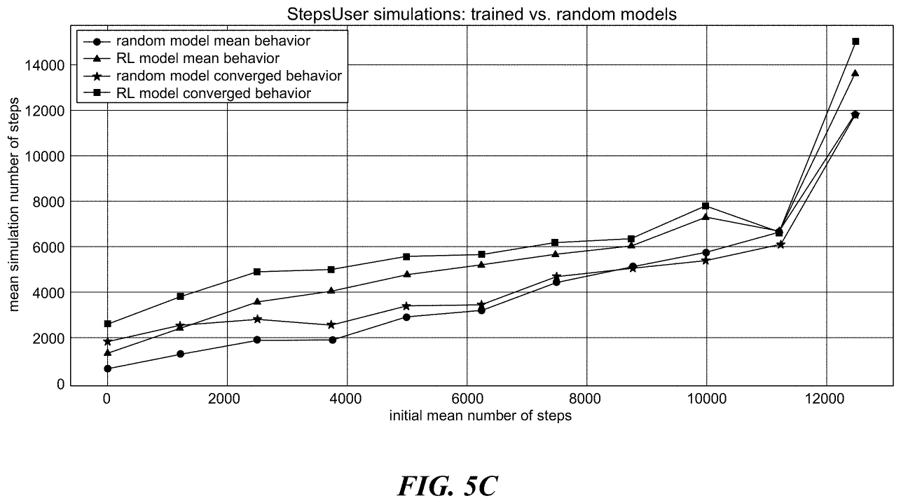

FIG. 5A-FIG. 5C are graphs illustrating comparisons between effects of using trained and random models in different simulations in accordance with some embodiments of the present technology. In FIG. 5A, a graph showing a plot of the average mean behavior and the end mean behavior from the random trained models for the reinforced user class as a function of the initial random mean behavior of the users is shown. The averages can correspond to 5000 episodes, each for 3000 steps. The results from reinforced user class simulations can show that, when using the trained model, the overall average behavior is 4.1 times higher than when using random actions, and that users probability of behavior converges to be 31 higher on average using the model than when using random actions. Because of the update dynamics of this type of users, in which the probability of behaving decreases by a fixed factor at each step, users' probability of behavior using the random model many times converges to very low values close to zero. While this happens when no directed intervention is available, when using the trained model most users can escape the decrease in mean behavior over time with the help of a policy that provides them with reinforcement at crucial points.

Similarly, a graph showing a plot of those means for the prompted user class is illustrated in FIG. 5B and a graph showing a plot of those means for the steps user is illustrated in FIG. 5C. In FIGS. 5B and 5C, the maximal number of steps is capped at 25,000 a day, in line with the 99^{th} percentile value in the data extracted from existing users.

The results for prompted user class simulations show a similar pattern to the reinforced user class simulations. Overall average behavior is 2.2 times higher than when using random actions, and that user probability of behavior converges to be 3.5 higher on average using the model than when using random actions.

Similarly, the results for steps user class simulations can show the overall average number of steps is 70.4% higher than when using random actions, and that users expected mean number of steps to which they converge at the end of the simulation can be 62% higher on average using the model than when using random actions.

The state vector used in the reinforcement learning algorithm can be extended to include other information. Information that changes frequently (e.g., time of day, location, biometric signals (such as temperature, heart rate, blood glucose, etc.), and/or self-reported information (such as food consumed, mood, etc.)) can all be incorporated into the state vector, so the model can learn how these different factors affect the individual's response to the actions. Information that changes infrequently or remains constant (e.g., gender, diagnosed conditions, or age) may have a diminished or no day-to-day effect on an individual's responsiveness to supports. However, in the pooled model context, it can still be useful to add such information to the state vector, so that the model training process can recognize similar patterns, if they appear in the data, of responsiveness in different states among individuals with similar quasi-constant characteristics.

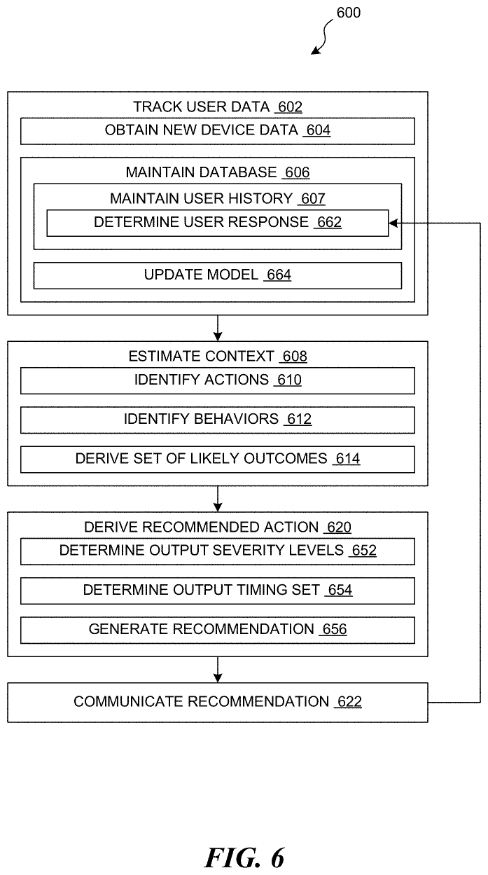

FIG. 6 is an example flow diagram for determining a best action for a user in accordance with embodiments of the present technology. The illustrated flow diagram can correspond to a method 600 of operating the healthcare guidance system 100 using an adaptive support model. Generally, the adaptive support model calculates an optimal action to be taken by a mobile application, such as whether or not, when, and/or how to prompt the user/patient, reinforce the user behavior, and/or present a daily goal to the user.

The system components can include a database (e.g., the database 106 of FIG. 1), a state estimator (e.g., the state estimator 114 of FIG. 1), and an adaptive support model (e.g., a portion of the models 122 of FIG. 1). The system components can be software components, hardware components, and/or hybrid software-hardware components used to implement the data flow.

At block 602, the system 100 can track user data. In tracking the user data, the system 100 can obtain new user data (e.g., the biometric data, the contextual data, the acceleration, the orientation data, etc.) from the user devices 104 of FIG. 1 as illustrated at block 604. The system 100 obtain and communicate the data between the devices in real-time and/or according to timings/events as described above. The system 100 can further maintain the database as illustrated at block 606. For example, the system 100 can maintain the user history, such as illustrated at block 607, by storing the obtained data in the user history 124 of FIG. 1. Other aspects of maintaining the database will be discussed in detail below.

The data flow retrieves a history of actions taken by the mobile application to support the user and resulting behavior of the user, collectively called user history, from the database. Other information related to the effect of the action on the behavior can be collected from the database as well, such as time elapsed between action taken and associated behavior being performed.

The data flow then estimates the user state (e.g., activities and/or health states) using state estimator 114. For example, the system 100 can estimate the context of the user as illustrated at block 608. Estimating the context may include estimating current and upcoming states (e.g., the health states of the user).

At block 610, the system 100 can identify individual actions of the user. The system 100 can compare the obtained data to predetermined templates to identify the current or last-performed action. For example, the system 100 can use changes in blood glucose level, user location, movement patterns, time of measurements, or a combination thereof to identify food intake action. Also, the system 100 can use changes in heart rate, user location, movement patterns, time of measurements, or a combination thereof to identify an exercise event. The system 100 can compare the data patterns to previous records and/or use the previous records to update the templates, thereby adapting the action identification to the individual user and/or progress of the user. The system 100 can record the actions in the user history.

At block 612, the system 100 can identify behaviors of the user. The system 100 can identify each behavior as a set of one or more repeated action as discussed above. The system 100 can use predetermined interval, frequency, and/or minimum quantity as thresholds for identifying the behaviors. The system 100 can update the user history to identify or categorize recorded data according to the identified behaviors.

At block 614, the system 100 can derive a set of likely outcomes given the currently received or accessible set of user data. The system 100 can derive the set of likely outcomes based on analyzing the most-current set of data and the user history, such as using the identified actions and behaviors.

The history of actions experienced by the user and the resulting behaviors performed by the user can be input into the state estimator 114. In some implementations, the input can also include parameters associated with the user including a set of values that correspond to likely user reactions to different stimuli. However, these parameters are not normally publicly available to the adaptive support model. When estimating the user's state, having at least an estimate of the likely user reactions is crucial. To obtain these parameters, a constrained minimization method can be used to find a maximum likelihood estimation of the parameters given the user history.

To obtain the maximum likelihood estimation (MLE) of the likely user reaction to different stimuli, a negative log likelihood function can be used, such as the function shown in Equation 2.

MLE=−log(P(b̅|θ,ā)) Equation 2:

In Equation 2, ‘b’ represents a sequence of behaviors, ‘a’ is a sequence of actions, and ‘0’ is the user parameters. The system 100 may operate on an assumption that behaviors are independent given the user parameters, the user state, and the previous behaviors and prior actions taken. Because the user state can be obtained via the parameters, previous behaviors, and past actions, Equation 2 simplifies to Equation 3.

MLE=−Σᵢ log P(bᵢ|θ,aᵢ,b_{0 . . . i-1}) Equation 3:

In Equation 3, bis a particular past behavior of the individual and aᵢ is a particular past action taken for the individual. Using Equation 3, the probability of seeing the particular past behavior bᵢ given at step i can be obtained. In some embodiments, the system 100 can use a threshold probability to derive the set of likely outcomes.

The data flow also includes identifying the best application action using the adaptive support model. At block 620, the system 100 can derive recommended actions based on the estimated context. The adaptive support model receives the estimated state (e.g., the user health state and/or the current or last-performed action) of the user from the state estimator and determines the likely effect on long term individual behavior from each possible action available for the mobile application to take. From these actions, the most beneficial action is selected. In some implementations, the most beneficial action can be determined based on one or more scores for the actions as described above. The most beneficial action, such as prompting the user, reinforcing the user, and the like, is then performed by the mobile application as illustrated at block 622.

The data flow also includes observing the resulting user behavior (e.g., user response) to the selected action. The performance (or nonperformance) of the targeted behavior associated with the selected action is recorded and saved to the database. In some implementations, the user's behavior is monitored via the mobile application, such as monitoring a response to a prompt given to a user. For example, a user can be prompted to immediately go on a walk, and the mobile application can track the user's location to determine if the user has gone on the walk (performed the behavior) or not (did not perform the behavior). In some implementations, the mobile application can also track a degree to which the behavior conforms to the action. For example, if the user is asked to take 10,000 steps during the day, the mobile application can track a number of steps the user takes during the day and can compare the number of steps to the 10,000 step threshold. If the user meets the threshold, the user performed the behavior. Otherwise, the user did not perform the behavior or only partially performed the behavior. This performance or non-performance of the behavior is then stored in the database for the user history. The recorded performances can be analyzed to adjust a severity and/or a communication timing (e.g., a duration offset of preceding the likely thresholding event) for the recommended action.

In some embodiments, each recommended action can include a category and/or a predetermined rating or measure representative of intensity, urgency, or magnitude. Further, the system 100 can retain the processing results (e.g., current and upcoming states, the corresponding timings, etc.) associated with the recommended actions. The system 100 can analyze these parameters similarly as described above to calculate the probability of seeing a targeted response given the category and/or the rating/measure. The system 100 can analyze the probability of seeing the targeted user response given the category and/or intensity of the recommended action. Accordingly, in deriving the recommended action, the system 100 can determine output severity levels as illustrated at block 652 and/or determine an output timing set as illustrated at block 654. The system 100 can use a process that corresponds to Equation 2 and Equation 3 described above. The system 100 can further evaluate the probability of targeted response based on the time between the recommendation and the user response, the degree of conformity in the user response, or a combination thereof. At block 656, the system 100 can generate the recommendation based on selecting the output severity level and/or the output timing having the highest probability of user conformance.

As an illustrative example, the blood glucose level of the user at 8:00 pm may be abnormal due to a current context of the user, such as missed meals, alcohol consumption, etc. The system 100 can further determine the user sleep behavior based on repeated resting patterns that begin within a threshold window around 11:00 pm. The system 100 can use these data values and behaviors to calculate a probability that the blood glucose level may reach a dangerous level at 3:00 am while the user sleeps. In deriving the corresponding recommended action, the system 100 can determine low scores for output timings that occur past 11:00 pm. Further, the system 100 can determine that the user historically responds best to urgent warnings and/or preventative actions between 10:00 pm to 11:00 pm (e.g., as part of bed-time routine). Accordingly, the system 100 can generate the recommendation corresponding to urgent warnings and/or higher incentives between 10:00 pm and 10:30 pm. Alternatively, if the user history indicates higher likelihood of user compliance for suggestions and/or relatively longer response durations, the system 100 may generate corresponding recommendations as soon as possible so that the user may comply before going to bed.

The data flow also includes updating the adaptive support model based on the performance or non-performance of the behavior, an estimated new state of the user, an action taken by the software application for the user, and other factors. After communicating the recommendation, the system 100 can analyze the incoming data to determine user response to or compliance with the recommended action as illustrated at block 662. When the user complies, the system 100 can assign corresponding positive scores or weights to the preceding recommendation. Negative or lower scores may be assigned for non-response or partial responses. The system 100 can use the response or a lack thereof to update one or more of the models as illustrated at block 664.

As described above, the system 100 can transform a variety of measurements and indications from one or more devices to estimated contexts, user health states, and likely future outcomes. The transformed parameters can be used identify a corrective action and details for communicating such actions in a way having the highest likelihood of user response. Accordingly, the current data and the user history may be further transformed into probability measures used to increase effectiveness in assisting the behavioral adjustments. The corresponding user responses can be identified and recorded into the user history, which can lead to further updates in one or more models. The updated models can be used to increase likeliness of the user response (e.g., the effectiveness of recommendations) for subsequent events. Thus, the system 100 can increase the accuracy in modeling the specific user and adapt to progress and actual changes in the user behavior over time.

In some implementations, the system 100 can also determine an adaptive score for the risk of the patient developing a cardiovascular disease (“CVD”) within a time period (e.g., one or more year, such as ten years). This score can be known as the cardiovascular score (“CV score”) for a patient. The CV score can be used in conjunction with the adaptive support model or another deep learning model to provide information about the risk of developing CVD. In some implementations, new inputs that are not normally used with the adaptive support model can be used. For example, instead of using only discrete values as input for the model, continuous values representing known risks of CVD can be used as inputs for the model, which represent risk score changes over time as new inputs from one or more sensors are obtained.

The scoring system can use a heart score to help illustrate a user's heart health, and the CVD score for the user can have a correlation to the heart score, such as indicating that there is an increased risk (e.g., 10-15% increase in CVD risk) for each point of the heart score. In this correlation, values of points are compared to those persons without any risk factors, who have a score of zero points, and aa relative risk of 1 (1.12⁰). For someone with twenty points as a heart score, the risk can be, for example, 9.64 (1.12²⁰) times higher than the risk of CVD for someone without any risk factors.

One of the risk factors can be body mass index, which can be defined as weight in kilograms divided by the square of individual's height in meters. The risk associated with body mass index can be assigned, in some implementations, as no risk points when body mass index is below a threshold value, such as 26 kilograms/m². For higher BMIs, the BMI risk value be calculated according to equation

BMI Risk Value=(BMI−26)/1.7 equation 4:

The BMI Risk Value can be limited to a number of points, such as 8 total points. The BMI can be calculated for each new weight entry, or a subset of entries, because the height of the individual should be constant. Accordingly, the BMI score can be used to adjust the CV score.

Another factor can be blood pressure medication information. Blood pressure medication information can be used to reduce CV score, as active use of these medications can reduce the risk of CVD. For example, the CV score can be reduced by, for example, 2 points for people without diabetes taking these medications, and by 1 point for people with diabetes taking these medications. The reduction is point values can be different for different medications, dosage, and underlying health conditions.

Yet another factor can be a continuous score for diabetes. For example, if a person does not have diabetes, blood glucose level scores can be used to assign CV score points based on a set of stepwise functions that are used to calculate an amount of points based on the blood glucose levels (e.g., blood glucose levels over a period of time, such as a thirty day average). In some implementations, an upper bound of points that can be assigned for people without diabetes can be set at, for example, 12 points. For people with diabetes, a separate set of stepwise functions can be used to calculate risk scores, with possible point values at a higher upper bound than those without diabetes and with functions reflecting the higher risk of CVD for those with diabetes.

Other factors can include age/sex, family history, tobacco usage, stress, depression, physical activity, diet, physical measurements, and the like. U.S. application entitled PREDICTIVE GUIDANCE SYSTEMS FOR PERSONALIZED HEALTH AND SELF-CARE, AND ASSOCIATED METHODS, filed Jun. 3, 2021 (Attorney Docket No. 137553.8017.US01), listing Daniel Goldner et al. as inventors discloses methods for scoring risks, disease risk, etc. and is incorporated by reference.

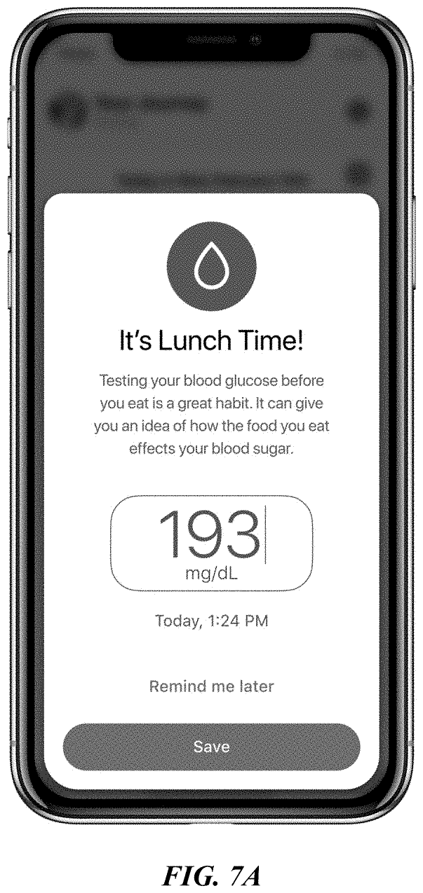

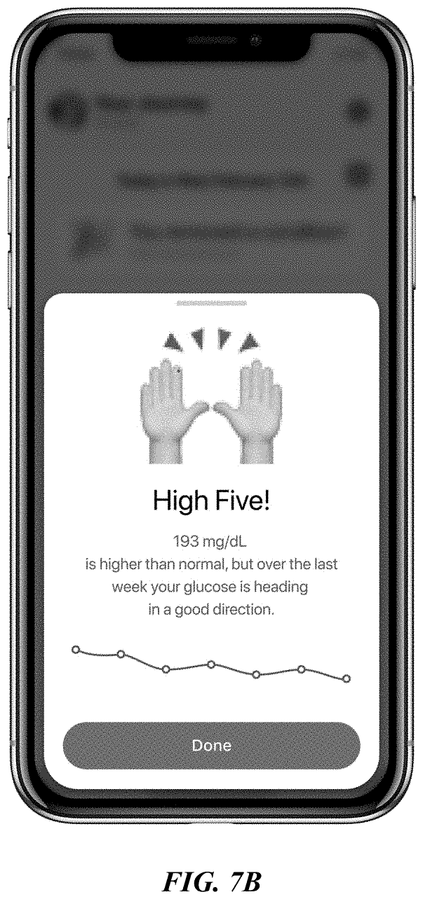

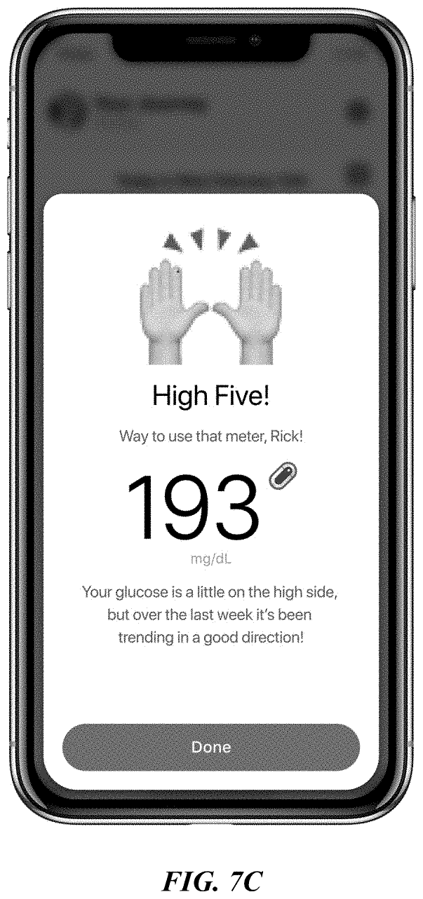

FIG. 7A-7C illustrate examples of prompts (FIG. 7A) and reinforcements (FIGS. 7B, 7C) output by a biomonitoring and healthcare guidance system configured in accordance with embodiments of the present technology. For example, a prompt 702 illustrated in FIG. 7A can include one or more predetermined messages or formats that correspond to a gentler or a less urgent message category. Also, reinforcements 704 and 706 of FIGS. 7B and 7C, respectively, can correspond to reward or positive feedback categories.

In some embodiments, the system 100 can adjust the messaging format according to user response. The system 100 can analyze the user responses as described above to show or increase the size of visual indicators (e.g., the downward graph of the reinforcement 704) for visually responsive users. Also, the system 100 can increase the size of measurement values (e.g., the blood glucose level in the reinforcement 706) for users that respond better to or focus more on numbers.

### Additional Embodiments

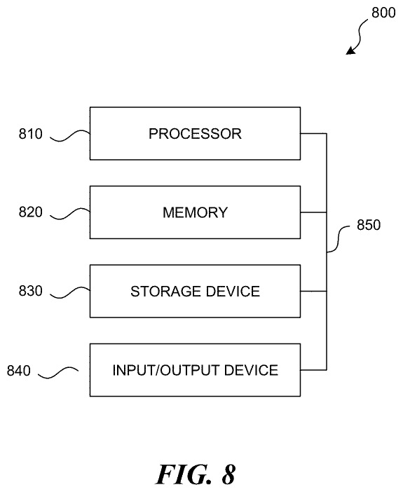

FIG. 8 is a schematic block diagram of a computing system or device (“system 800”) configured in accordance with embodiments of the present technology. The system 800 can be incorporated into or used with any of the systems and devices described herein, such as the analyzing devices 102 and/or user devices 104 of FIG. 1. The system 800 can be used to perform any of the processes or methods described herein with respect to FIGS. 1 and 2. The system 800 can include a processor 810, a memory 820, a storage device 830, and an input/output device 840. Each of the components 810, 820, 830 and 840 can be interconnected using a system bus 850. The processor 810 can be configured to process instructions for execution within the system 800. In some embodiments, the processor 810 can be a single-threaded processor. In alternate embodiments, the processor 810 can be a multi-threaded processor. Although FIG. 8 illustrates a single processor 810, in other embodiments the system 800 can include multiple processors 810. In such embodiments, some or all of the processors 810 can be situated at different locations. For example, a first processor can be located in a sensor device, a second processor can be located in a user device (e.g., a mobile device), and/or a third processor can be part of a cloud computing system or device.

The processor 810 can be further configured to process instructions stored in the memory 820 or on the storage device 830, including receiving or sending information through the input/output device 340. The memory 820 can store information within the system 800. In some embodiments, the memory 820 can be a computer-readable medium. In alternate embodiments, the memory 820 can be a volatile memory unit. In yet some embodiments, the memory 820 can be a non-volatile memory unit. The storage device 830 can be capable of providing mass storage for the system 800. In some embodiments, the storage device 830 can be a computer-readable medium. In alternate embodiments, the storage device 830 can be a floppy disk device, a hard disk device, an optical disk device, a tape device, non-volatile solid state memory, or any other type of storage device. The input/output device 840 can be configured to provide input/output operations for the system 800. In some embodiments, the input/output device 840 can include a keyboard and/or pointing device. In alternate embodiments, the input/output device 840 can include a display unit for displaying graphical user interfaces.

Non-transitory computer program products (i.e., physically embodied computer program products) are also described that store instructions that, when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Alternatively or in combination, the display device can be a touchscreen or other user input device configured to accept tactile input (e.g., via a virtual keyboard and mouse). Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The technology described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

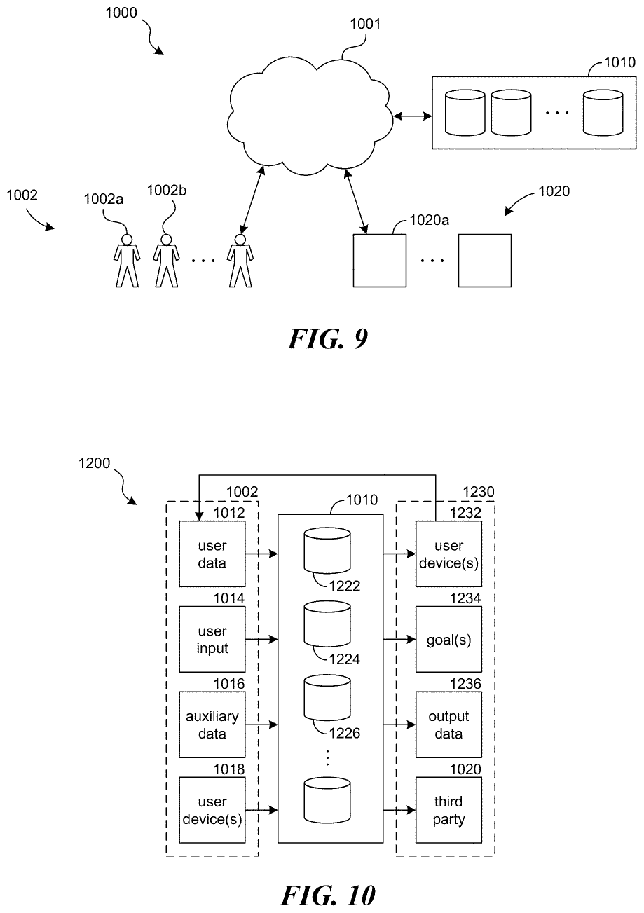

FIG. 9 is a schematic diagram illustrating an embodiment of the system for providing adaptive healthcare support, in accordance with embodiments of the present technology. A system 1000 can include a network 1001, a biomonitoring and healthcare guidance system 1010 (system 1010), users or user devices 1002 (“user devices 1002”), and additional systems 1020. The network 1001 can transmit data between the user devices 1002, healthcare guidance system 1010, and/or additional systems 1020. The system 1010 can select one or more databases, models, and/or engines to analyze received data. The description of system of 100 of FIG. 1 applies equally to the system 1000 unless indicated otherwise, and the system 1000 can perform the methods disclosed herein.

The system 1010 can include databases, models, systems, and other features disclosed herein and can include models, algorithms, engines, features, and systems disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; PCT App. No. PCT/2021/028445, and other patents and applications discussed herein. For example, the system 1010 can receive health data (e.g., glucose levels, blood pressure, etc.) from user devices disclosed in U.S. application Ser. No. 16/888,105 or U.S. application Ser. No. 17/236,753 and can forecast or predict one or more health metrics disclosed in U.S. application Ser. No. 16/888,105 or U.S. application Ser. No. 17/167,795. The forecasted metrics can be used to determine a behavioral intervention plan. The system 1000 can provide behavioral interventions to achieve exercise goals. For example, the user 1002b can be training to increase cardiovascular levels. The system 1000 can receive user exercise data (e.g., workout type, workout duration, etc.), exercise data (e.g., heart rate, blood pressure, etc.), positioning data (e.g., GPS data), or other data. The system 1000 can then determine healthcare support actions and behavioral interactions to be performed to, for example, develop behavioral intervention plan for completing workouts. The system 1010 can use forecasting models or engines to determine recommendations for the user and can generate new models based on newly available data. Forecasting models or engines can be used for multiple users or a single user. In some embodiments, data associated from a user can be inputted into different models or engines and the output from those engines or models can be grouped, processed, and/or feed into additional models or engines, including those disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; PCT App. No. PCT/2021/028445.

The network 1001 can communicate with devices or computing system 1020. The computing systems 120 can provide programs, or other information used to manage the collection of data. For example, a computing system 1022a can communicate with a wearable user device 1020a to provide firmware updates, OTA software updates, or the like. The guidance system 1010 can automatically update databases, models, and/or engines based on changes to the user device 1002a. The computing system 1022a and guidance system 1010 can communicate with one another to further refine data analysis.

A user can manage privacy and data settings to control data flow. In some embodiments, one of the computing systems 1020 is managed by the user's healthcare provider so that received user data is automatically sent to the user's physician. This allows the physician to monitor the health and progress of the user. The physician can be notified of changes (e.g., health-related events) to provide further reinforcement monitoring. The guidance system 1010 can adjust behavioral interventions based on input from the healthcare provider. For example, the healthcare provide can add health care support parameters, such as target goals for losing weight, reducing blood pressure, increasing exercise durations, etc. The behavioral intervention programs can be modified by the user, healthcare provider, family member, authorized individual, etc.

The healthcare guidance system 1020 can forecast events, predict health states, and/or perform any of techniques or methods disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445. For example, the system 100 can accurately determination of glucose concentration in the blood of an individual at a present time and/or in the future and can adaptively provide healthcare support to achieve health goals. The system 1000 can then develop personalized biomonitoring and/or providing personalized healthcare recommendations or information for the treatment of diabetes and other chronic conditions, exercise programs, or the like.

FIG. 10 is a schematic diagram illustrating an embodiment of the system for providing adaptive healthcare support for a user 1002, in accordance with an embodiment of the present technology. The description of the system 1000 of FIG. 10 applies equally to the system 1200 unless indicated otherwise.

The system 1200 can collect user data, user input, auxiliary data, etc. The user data can be collected by sensors (e.g., glucose sensors, wearable sensors, etc.), received from a remote computing device (e.g., a cloud platform storing user history data, real-time data, etc.), or other data. The user input can be health data (e.g., weight, BMI, etc.), exercise or motion data (e.g., distance walked, distance run, etc.), goals, achievements, ratings/rankings (e.g., ranked goals, rated activities, etc.), or other data inputted by the user using one or more computing devices, such as a mobile phone, computer, etc. This allows a user in input data that is not automatically collected. The auxiliary data 1216 can be selected by the system 1210 to modify the adaptive support machine-learning model based on received indication of the response. The auxiliary data 1216 can include predictions (e.g., short-term predictions, long-term predictions, forecasted events, etc.), environment data (e.g., weather data, temperature data, etc.), or the like. The auxiliary data 1216 can be inputted to models to generate output data based on non-user specific parameters.

The system 1010 can request auxiliary data or communicate with device(s) to receive data indicative of a past user state, a past action presented to the user, a past user behavior, health status, or combinations thereof. In some embodiments, the system 1010 can establish communication with connected device (e.g., vehicle) associated with the user, IoT hubs (e.g., IoT devices with Google Assistance, Siri, Alexa, etc.), IoT devices (e.g., motion sensors, cameras, etc.), surveillance systems, etc. For example, when a user arrive home after work, the user may not be receptive to certain prompts for a period of time. The system 1010 can receive auxiliary data (e.g., a garage door opening, surveillance system turned OFF, etc.) indicating when the user returned home. The system 1010 can determine a program or a set of delivery details for adjusting a content and/or a delivery timing for recommended actions based on the user's arrival time. The system 1010 can adaptive request and receive data from different sources to adaptive train the models and engines disclosed herein. The system 1010 can manage identification and authentication for integration with auxiliary platforms, devices, and systems. In some applications, the system 1010 can incorporate weather data to maximize behavior intervention by, for example, providing prompt (e.g., prompts to exercise outside, walk, etc.) suitable for the weather conditions. Health predictions can be considered to develop behavioral interventions designed to increase health scores for the user.

The user input 1014 can include one or more new goals, such as maintaining glucose levels, losing weight within a set period or time, etc. The guidance system 1010 can select databases (e.g., pooled user data) and models for recommending user device(s) for collecting target data, analyzing the one or more new goals, recommending user device(s) for reinforcements, etc. The guidance system 1010 can send the information to user device 1232 for viewing by a healthcare provider or third-party device 1238, as discussed in connection with FIG. 10.

The system 1010 can receive one or more user history items associated with the user 1002. The user history items can define a past user state, a past action presented to the user, a past user behavior, or combinations thereof. The system 1020 can select an adaptive healthcare support engine 1222 trained to estimate user information, such a current state or predicted state of the user, based on the one or more user history items. The system 1020 can utilize the adaptive healthcare support engine 1222 or another engine 1224 to identify one or more actions for the user based on the user information. The user device(s) 1018, 1232 can execute the one or more identified actions for the user and can receiving an indication of a behavior of the user performed in response to the action. The system 1020 can update one or more of the adaptive support models (e.g., models 1222, 1224, etc.) based on the received indication of the behavior detected by the user devices 1018 or 1232, or indicated by user 1014.

In some embodiments, the system 1010 can receive new data from the user 1002. The new data can represent health sensor data, a biometric condition, user input data, a user motion, a user location, or a combination thereof. The health sensor data from a user device 1018 can include glucose levels, blood pressure, heart rate, analyte levels, or other detectable indicators of the state of the user. The system 1010 can access one or more user history items (e.g., items stored in database 1226) defining at least one of a past user state, a past action presented to the user, and a past user behavior. The past user state can represent a physiological or a health condition of the user occurring or processed at a past time. The past action can represent a previously identified action taken by the user. The past user behavior can represent a repeated action occurring with a temporal pattern. The actions can be detected or identified by user device(s) 1018, 1232, or another suitable means, such as biomonitoring devices or via user input 1014.

The system 1010 can estimate a recent state of the user based on the new data and one or more user history items. The recent state represents a current or a recent health condition of the user (e.g., most recent health condition, health condition within a predetermined period of time, etc.). The health condition can be, for example, hypoglycemic, hyperglycemic, high blood pressure, etc. The system 1010 can determine a likely outcome (e.g., increase/decrease in glucose levels, blood pressure, etc.) based on the recent state for represent a thresholding health condition of the user likely to occur at a future time. The system 1010 can then identifying one or more actions for the user based on the recent state using one or more adaptive support machine-learning models. The actions can be sent to the user devices 1232 for user notification to affect a targeted user action before the future time to prevent or adjust the likely outcome. In some embodiments, the identified actions are selected based on whether the user devices 1232 is capable of identifying the action. For example, if the user has wearable exercise monitor, the identified actions can include exercises detectable by the wearable exercise monitor. In some embodiments, the user can be prompted to input whether the action has been completed. The 1010 can also provide goal(s) 1234, output data 1236, or other information disclosed in U.S. application Ser. No. 14/812,288; U.S. Pat. Nos. 10,820,860; 10,595,754; U.S. application Ser. No. 16/558,558; PCT. App. No. PCT/US2019/049270; U.S. application Ser. No. 16/888,105; PCT App. No. PCT/US20/35330; U.S. application Ser. No. 17/167,795; U.S. application Ser. No. 17/236,753; and PCT App. No. PCT/2021/028445.

The system 1010 can also determine a set of delivery details for adjusting a content and/or a delivery timing for the recommended action. The user device(s) 1232 can execute the identified action according to the set of delivery details. The system 1200 can receive or identify and indication of a response of the user performed in response to the action. When the response corresponds to the past user behavior, the system 1010 can update associated adaptive support machine-learning models based on the received indication of the response. The system 1010 can add engines and models based on newly available data, new users, or the like to provide adaptability.

### CONCLUSION

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the embodiments described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments can be within the scope of the following claims.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

Furthermore, the skilled artisan will recognize the interchangeability of various features from different embodiments disclosed herein and disclosed in U.S. Pat. Nos. 9,008,745; 9,182,368; 10,173,042; U.S. application Ser. No. 15/601,204 (US Pub. No. 2017/0251958); U.S. application Ser. No. 15/876,678 (U.S. Pub. No. 2018/0140235); U.S. application Ser. No. 14/812,288 (US Pub. No. 2016/0029931); U.S. application Ser. No. 14/812,288 (US Pub. No. 2016/0029966); US Pub. No. 2017/0128009; U.S. App. No. 62/855,194; U.S. App. No. 62/854,088; U.S. App. No. 62/970,282; U.S. 63/034,333; PCT App. No. PCT/US19/49270 (WO2020/051101); U.S. application Ser. No. 17/236,753; PCT App. No. PCT/2021/028445; and U.S. Application entitled PREDICTIVE GUIDANCE SYSTEMS FOR PERSONALIZED HEALTH AND SELF-CARE, AND ASSOCIATED METHODS, filed Jun. 3, 2021 (Attorney Docket No. 137553.8017.US01), listing Daniel Goldner et al. as inventors. For example, methods of detection, sensors, detection elements, biosensors, user devices, etc. can be incorporated into or used with the technology disclosed herein. Similarly, the various features and acts discussed above, as well as other known equivalents for each such feature or act, can be mixed and matched by one of ordinary skill in this art to perform methods in accordance with principles described herein. All of the above cited applications and patents are herein incorporated by reference in their entireties.

From the foregoing, it will be appreciated that specific embodiments of the invention have been described herein for purposes of illustration, but that various modifications may be made without deviating from the scope of the invention. Accordingly, the invention is not limited except as by the appended claims.

1. A method for operating a health guidance system, the method comprising:
obtaining new data from one or more user devices, wherein the new data represents a biometric condition, a user input, a user motion, a user location, or a combination thereof for a user;
accessing one or more user history items associated with the user, the user history items defining at least one of a past user state, a past action presented to the user, and a past user behavior, wherein
the past user state represents a physiological or a health condition of the user occurring or processed at a past time,
the past action represents a previously identified action taken by the user, and
the past user behavior represents a repeated action occurring with a temporal pattern;
estimating a recent state of the user based on the new data and the one or more user history items, wherein the recent state represents a current or a most recent health condition of the user;
estimating a likely outcome based on the recent state, wherein the likely outcome represents a thresholding health condition of the user likely to occur at a future time;
identifying an action for the user based on the recent state of the user using an adaptive support machine-learning model, wherein
the action represents an action performed by the health guidance system to affect a targeted user action before the future time to prevent or adjust the likely outcome, and
identifying the action includes identifying a set of delivery details for adjusting a content and/or a delivery timing for the recommended action;
executing the identified action for the user according to the set of delivery details;
receiving an indication of a response of the user performed in response to the action, wherein the response corresponds to the past user behavior; and
updating the adaptive support machine-learning model based on the received indication of the response.

2. The method of claim 1, wherein the adaptive support machine-learning model is a deep neural network model.

3. The method of claim 1, wherein the identified action is identified from a group of actions using a determined likely compliance value for each action and a corresponding set of delivery details, the determined likely compliance value for each action being generated by the adaptive support machine-learning model configured to assist the user in changing a user behavior over time.

4. The method of claim 1, wherein the user recent state is further estimated using parameters associated with the user.

5. The method of claim 4, wherein the parameters associated with the user are estimated using a maximum likelihood estimation function.

6. The method of claim 1, wherein the action is at least one of a prompt for encouraging the user to perform the targeted user action, a warning regarding the likely outcome, and a reinforcement for the user for performing the targeted user action.

7. The method of claim 1, wherein the received indication represents at least one of a performance of the targeted user action, a partial performance of the targeted user action, and non-performance of the targeted user action.

8. A computer-readable medium comprising instructions that, when executed by one or more processors, cause the one or more processors to perform a process, the process comprising:
obtaining new data from one or more user devices, wherein the new data represents a biometric condition, a user input, a user motion, a user location, or a combination thereof for a user;
accessing one or more user history items associated with the user, the user history items defining at least one of a past user state, a past action presented to the user, and a past user behavior, wherein
the past user state represents a physiological or a health condition of the user occurring or processed at a past time,
the past action represents a previously identified action taken by the user, and
the past user behavior represents a repeated action occurring with a temporal pattern;
estimating a state of the user based on the new data and the one or more user history items, wherein the recent state represents a current or a most recent health condition of the user;
estimating a likely outcome based on the recent state, wherein the likely outcome represents a thresholding health condition of the user likely to occur at a future time;
identifying an action for the user based on the state of the user using an adaptive support model, wherein
the adaptive support model is a machine-learning model,
the action represents an action performed by the health guidance system to affect a targeted user action before the future time to prevent or adjust the likely outcome, and
identifying the action includes identifying a set of delivery details for adjusting a content and/or a delivery timing for the recommended action;
executing the identified action for the user according to the set of delivery details;
receiving an indication of a response of the user performed in response to the action, wherein the user action corresponds to the past user behavior; and
updating the adaptive support model based on the received indication of the response.

9. The computer-readable medium of claim 8, wherein the adaptive support model is a deep neural network model.

10. The computer-readable medium of claim 8, wherein the identified action is identified from a group of actions using a determined likely compliance value for each action and a corresponding set of delivery details, the determined likely compliance value for each action being generated by the adaptive support model configured to assist the user in changing a user behavior over time.

11. The computer-readable medium of claim 8, wherein the user state is further estimated using parameters associated with the user.

12. The computer-readable medium of claim 11, wherein the parameters associated with the user are estimated using a maximum likelihood estimation function.

13. The computer-readable medium of claim 8, wherein the action is at least one of a prompt for encouraging the user to perform the targeted user action, a warning regarding the likely outcome, and a reinforcement for the user for performing the targeted user action.

14. The computer-readable medium of claim 8, wherein the received indication represents at least one of a performance of the targeted user action, a partial performance of the targeted user action, and non-performance of the targeted user action.

15. A computing system comprising:
one or more processors; and
memory having stored thereon instructions that, when executed by the one or more processors, cause the one or more processors to perform a process, the process comprising:
obtaining new data from one or more user devices, wherein the new data represents a biometric condition, a user input, a user motion, a user location, or a combination thereof for a user;
accessing one or more user history items associated with the user, the user history items defining at least one of a past user state, a past action presented to the user, and a past user behavior, wherein
the past user state represents a physiological or a health condition of the user occurring or processed at a past time,
the past action represents a previously identified action taken by the user, and
the past user behavior represents a repeated action occurring with a temporal pattern;
estimating a state of the user based on the new data and the one or more user history items, wherein the recent state represents a current or a most recent health condition of the user;
estimating a likely outcome based on the recent state, wherein the likely outcome represents a thresholding health condition of the user likely to occur at a future time;
identifying an action for the user based on the state of the user using an adaptive support model, wherein
the adaptive support model is a machine-learning model,
the action represents an action performed by the health guidance system to affect a targeted user action before the future time to prevent or adjust the likely outcome, and
identifying the action includes identifying a set of delivery details for adjusting a content and/or a delivery timing for the recommended action;
executing the identified action for the user according to the set of delivery details;
receiving an indication of a response of the user performed in response to the action, wherein the user action corresponds to the past user behavior; and
updating the adaptive support model based on the received indication of the response.

16. The computing system of claim 15, wherein the identified action is identified from a group of actions using a determined likely compliance value for each action and a corresponding set of delivery details, the determined likely compliance value for each action being generated by the adaptive support model configured to assist the user in changing a user behavior over time.

17. The computing system of claim 15, wherein the user state is further estimated using parameters associated with the user.

18. The computing system of claim 17, wherein the parameters associated with the user are estimated using a maximum likelihood estimation function.

19. The computing system of claim 15, wherein the action is at least one of a prompt for encouraging the user to perform the targeted user action, a warning regarding the likely outcome, and a reinforcement for the user for performing the targeted user action.

20. The computing system of claim 15, wherein the received indication represents at least one of a performance of the targeted user action, a partial performance of the targeted user action, and non-performance of the targeted user action.

21-40. (canceled)
